(19) United States
(12) Patent Application Publication
(10) Pub. No.: US 2026/0096766 A1
(43) Pub. Date: Apr. 9, 2026

(54) SYSTEM AND METHODS FOR TREATING NEUROPATHOLOGIES OF A SUBJECT

(71) Applicant: THE CLEVELAND CLINIC FOUNDATION, Cleveland, OH (US)

(72) Inventor: Francis A. Papay, Westlake, OH (US)

(21) Appl. No.: 19/112,917

(22) PCT Filed: Sep. 20, 2023
(86) PCT No.: PCT/US2023/033246

§ 371 Date: Mar. 18, 2025

Related U.S. Application Data
(60) Provisional application No. 63/408,280, filed on Sep. 20, 2022.

(51) Int. Cl. A61B 5/294; A61B 5/268; A61N 1/05; A61N 1/36

(52) U.S. Cl. CPC A61B 5/294; A61B 5/268; A61N 1/0551; A61N 1/3611

(57) ABSTRACT

A method of treating an animal subject includes applying a nerve interface to a muscle pedicle of the subject, the muscle pedicle having a peripheral nerve inserted therein, wherein the nerve interface is configured to provide electrical communication between the peripheral nerve and an electronic device.

### CROSS-REFERENCE TO RELATED APPLICATIONS

This application claims the benefit of U.S. provisional application Ser. No. 63/408,280 filed Sep. 20, 2022, the contents of which are incorporated by reference.

### FIELD OF THE INVENTION

The present disclosure relates to a system and methods for treating neuropathologies of a subject and more particularly, neuropathologies such as sleep apnea or oral pharyngeal paralysis.

### BACKGROUND

Bioelectricity is a broad field including measurements of biopotentials and bioimpedance. Biopotentials relate to electricity created in life processes within biologic tissues, while bioimpedance is the response of a living organism to an externally applied electric current. Biopotentials thus generally refer to active processes, such as excitation of nerve and muscle tissues, whereas bioimpedance is related to passive properties of the tissue in response to an external electrical stimulus, such as the properties of the skin. The passive bioimpedance properties can also be related or correlated to electrical or other processes within biologic tissues, even though measurement of bioimpedance does not directly utilize the electricity generated within tissues.

Electromyography (EMG) is a method for recording electrical biopotentials of muscles. In an EMG measurement, electrodes are attached under the skin at a particular muscle grouping such as the superior strap muscles of the neck (Geniohyoid, Mylohyoid, and more). An EMG signal is recorded from the face and neck of the patient, whereby the recorded signal both indicates the activity of the facial and neck muscles (FNEMG) and may be related to other electrical activity studies of the brain (EEG). As the frequencies of the EMG spectrum are usually high and above the frequencies of the brain activity, the signal components can be separated by methods of signal processing or spectral analysis from the EMG signal as they relate to certain targeted activities of the brain.

### BRIEF SUMMARY OF THE INVENTION

According to a first aspect, a method of treating an animal subject includes applying a nerve interface to a muscle pedicle of the subject, the muscle pedicle having a peripheral nerve inserted therein, wherein the nerve interface is configured to provide electrical communication between the peripheral nerve and an electronic device.

According to second aspect, a method of treating an animal subject includes applying a nerve interface to a cranial or cervical nerve. The nerve interface includes a main body, an electrode affixed to the main body, and one or more conductive polymers that establish electrical communication between the peripheral nerve and the electrode. Moreover, the method includes monitoring neuroactivity of the cranial or cervical nerve, or applying electrical stimulus to the cranial or cervical nerve, via the nerve interface.

According to a third aspect, a nerve interface device for treating a subject includes an electrical interface configured to be applied to a muscle pedicle of an animal subject, the muscle pedicle having a peripheral nerve inserted therein, wherein the nerve interface is configured to provide electrical communication between the peripheral nerve and an electronic device.

### BRIEF DESCRIPTION OF THE DRAWINGS

FIG. 1 shows an example system for treating neuropathologies of a patient.

### DETAILED DESCRIPTION

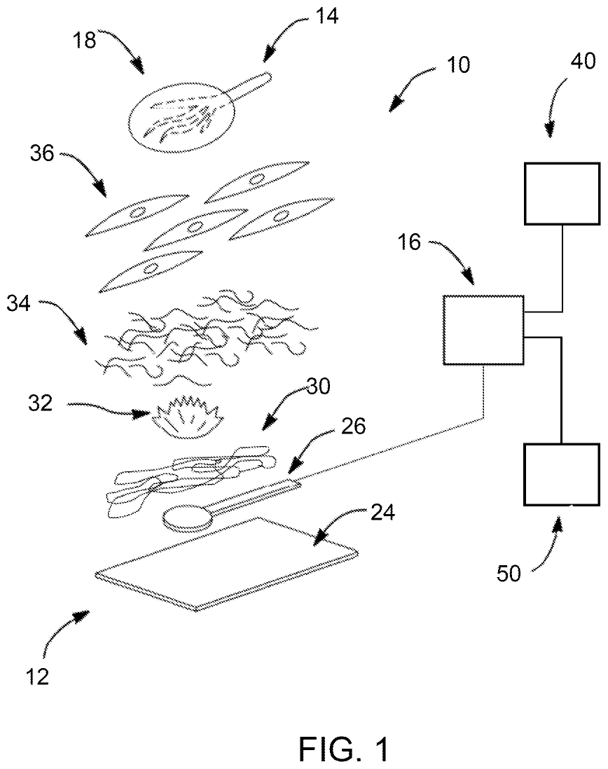

Turning to FIG. 1, an example system 10 for treating neuropathologies of a patient will now be described. The system 10 includes an implantable nerve interface 12 that can be applied to a peripheral nerve 14 of an animal (e.g. human) subject, and an electronic device 16 that can be electrically connected to the interface 12. As discussed further below, the interface 12 can establish communication between the nerve 14 and electronic device 16, such that the system 10 can be used to monitor neuroactivity and/or apply electrical stimulus to the nerve. The system 10 can be particularly useful when applied to cranial or cervical nerves in the skull-base and neck region, for treating neuropathologies such as sleep apnea or oral pharyngeal paralysis.

In the present example, the nerve 14 corresponds to the ansa hypoglossi nerve of the subject. Moreover, the nerve interface 12 is applied to a muscle pedicle 18 of the subject, which is formed by dissecting a portion of muscle having the nerve 14 inserted therein. In the current example, the muscle pedicle 18 is formed by dissecting a strap muscle (e.g., the sternohyoid muscle) of the anterior neck at a location in which the ansa hypoglossi nerve 14 is inserted. However, the nerve interface 12 can be applied to other muscle portions and nerves without departing from the scope of the disclosure. Other examples include a nerve-muscle pedicle of the cranial nerves (Cranial Nerves 5-12) and/or cervical nerve units for electrical signal trigger to initiate neuromodulation or neurostimulation of the craniofacial musculature, cervical musculature, lingual/pharyngeal musculature, or phrenic/diagphragmatic nerve muscle unit. Moreover, in some examples, the nerve interface 12 can be applied directly to the nerve 14 without forming a muscle pedicle. For example, the nerve 14 can be separated or exposed from an associated muscle, and the nerve interface 12 can be directly affixed to the nerve 14. Preferably, the nerve 14 corresponds to a motor nerve of the subject, although it may also be a sensory nerve in some examples.

The nerve interface 12 includes a main body 24 and an electrode 26 affixed to the main body 24. Moreover, various layers of materials are applied over the electrode 26 including a layer of scaffold material 30, a first conductive polymer 32, a second conductive polymer 34, and a layer of autograft of muscle tissue 36. The materials 30-36 are layered such that the scaffold material 30 covers the electrode 26, the first conductive polymer 32 covers the scaffold material 30, the second conductive polymer 34 covers the first conductive polymer 32, and the autograft of muscle tissue 36 covers the second conductive polymer 34. Each layer 30-36 directly contacts its adjacent layer 30-36 below, and may also contact layers further below. For example, the second conductive polymer 34 directly contacts the first conductive polymer 32, and may also contact portions of the scaffold material 30 and electrode 26. Moreover, it is to be appreciated that the nerve interface 12 may include additional or fewer layers in other examples.

The main body 24 in the present example is an electrically insulating substrate that supports the electrode 26 and layers 30-36 above. The main body 24 can comprise an insulating material such as poly-paraxylylene (parylene), polyimide, silicon dioxide, or combinations thereof. In some examples, the main body 24 can be wrapped around the muscle pedicle 18 to form a tubular body that encloses the pedicle 18 therein against the adjacent muscle-tissue layer 36, with the subjacent electrode 26 and layers 30-32 disposed intermediate the main body 24 and the enclosed pedicle 18. In other examples, the main body 24 can be a pre-formed tubular housing with an opening for receiving one or more of the elements 18, 26, 30-36.

The electrode 26 comprises a metallic material having metal-like properties that are desired in appropriate applications. The metallic material can comprise Gold (Au), Platinum (Pt), Iridium (Ir), Palladium (Pd), Tungsten (W), Stainless Steel (SS), Indium-Tin-Oxide (ITO), Zinc (Zn), Titanium (Ti), or combinations thereof. In other variations, the electrode 26 may comprise a semiconductor material such as Carbon (C), Silicon (Si), alloys, oxides, nitrides, or combinations thereof. Thus, the electrode 26 may comprise a metal material or a semiconductor material selected from a group consisting of: Gold (Au), Platinum (Pt), Iridium (Ir), Palladium (Pd), Tungsten (W), Stainless Steel (SS), Indium-Tin-Oxide (ITO), Zinc (Zn), Titanium (Ti), Carbon (C), Silicon (Si), alloys, oxides, nitrides, and combinations thereof.

Still further, the electrode 26 can be coated with a non-metallic material such as poly(3,4-ethylenedioxythiophene) (PEDOT), poly(pyrrole), polyaniline, polyacetylene, polythiophene, natural or synthetic melanin, their derivatives, or combinations thereof. Electrodes coated with PEDOT electroconductive polymer can yield a lower impedance and higher charge density than uncoated stainless-steel electrodes. PEDOT-coated electrodes have more robust cyclic voltammetry than uncoated stainless-steel electrodes, which allows for a greater range of applied current. Additionally, PEDOT-coated electrodes have a decreased rheobase. PEDOT-coated electrodes are more sensitive and have a higher fidelity than uncoated electrodes. BT-DOT (Biotectix, Ann Arbor, Mich.) may also be used as the non-metallic material.

The layer of scaffold material 30 can comprise a decellularized biotic material (e.g., small intestinal submucosa (SIS)), a hydrogel, a biological scaffold material, a biocompatible polymeric material, a cellularized biotic or abiotic material, or combinations thereof. A biotic material can be sourced from biologic, biologically-derived, or bio-functionalized material. In certain aspects, the biotic material may be an acellularized tissue scaffold, any type of biological scaffold (e.g., a collagen matrix and the like), or a cellularized biotic or abiotic scaffold (if cells are embedded in the scaffold prior to implantation). The biotic material can include autologous, allogous, or allogeneic or xenogeneic tissue, preferably tissue capable of supporting the growth of neural tissue, including neurons and substructures thereof, skeletal muscle, cardiac muscle, smooth muscle, and cells thereof. In some embodiments, the biological component can contain a plurality of cells derived from autologous, allogous, or allogeneic or xenogeneic tissue sources. Moreover, it is to be appreciated that the main body 24 can comprise similar materials as the layer of scaffold material 30.

The first conductive polymer 32 can comprise poly(3,4-ethylenedioxythiophene) (PEDOT), poly(pyrrole), polyaniline, polyacetylene, polythiophene, natural or synthetic melanin, their derivatives, or combinations thereof. BT-DOT (Biotectix, Ann Arbor, Mich.) may also be used for the first conductive polymer 32. When BT-DOT is applied to the electrode 26 it significantly reduces the amount of current needed to evoke a muscle tissue response. In other aspects, the first conductive polymer 32 comprises poly(3,4-ethylenedioxythiophene) (PEDOT), which allows nerve signals from the biotic nerve tissue to cross a 20 mm graft of acellular tissue polymerized with PEDOT and continue to propagate through the distal end of the nerve.

The second conductive polymer 34 can comprise poly(pyrrole), polyaniline, polyacetylene, polythiophene, ester derivative, 3,4-propylenedioxythiophene (ProDOT), natural or synthetic melanin, their derivatives, or combinations thereof. In embodiments in which the layer of scaffold material 30 comprises small intestinal submucosa (SIS), the second conductive polymer 34 can be electrochemically polymerized through the SIS. The second conductive polymer 34 allows the supporting scaffold material 30 to remain compliant, thus preventing high sheer stresses in regions where soft, pliable nervous tissue contacts hard, rigid, inorganic materials. Other polymers and electrochemical polymerization techniques can be used that allow for low impedance conductive polymer microflowers, which functionally bridge from the microfabricated electrode through the SIS scaffold material 30.

The autograft of muscle tissue 36 can be resected from the subject and disposed over the second conductive polymer 34, thereby integrating into the porous conductive polymer-coated SIS to provide a stable, soft interface that is highly reliable, intimate, and mechanically compliant.

The electrode 26 of the interface 12 is configured to be in electrical communication with the nerve 14 via one or more of the layers 30-36 therebetween. Moreover, the electrode 26 can be electrically connected to the electronic device 16 (e.g., via one or more leads) to provide electrical communication between the nerve 14 and device 16. The nerve interface 12 is thus a multi-layered interface that can interface between the nerve 14 and electronic device 16. Moreover, the nerve interface 12 creates an environment where neurons can regenerate, reinnervate, and create stable, biologically active connections with muscle or sensory cells.

Furthermore, by applying the nerve interface 12 to the muscle pedicle 18, the intended muscle will be activated with the nerve during stimulation. This eliminates relying on native anatomy which can be different between individuals as a natural occurring phenomenon. A known target can lead to precise stimulation, reduce the amount and duration of stimulation, and improve patient outcome.

However, it is to be appreciated that the interface 12 may comprise other configurations without departing from the scope of the disclosure. In one example, the interface 12 can correspond to the nerve interface described in U.S. Pat. No. 9,352,146, which is hereby incorporated by reference in its entirety. Moreover, as noted above, the interface 12 may be applied directly the nerve 14 without forming a muscle pedicle. Broadly speaking, the nerve interface 12 can comprise any configuration that is applied to the nerve 14 and enables communication between the nerve 14 and electronic device 16.

The electronic device 16 may also be an implantable device that is surgically implanted in the subject with the interface 12. Alternatively, the electronic device 16 may reside outside of the subject and be operatively connected to the interface 12 via one or more leads that penetrate the body. The device 16 can comprise one or more electrical components such as a power supply (e.g., battery), a microprocessor, an electronic data storage unit (e.g., memory), a digital interface (e.g., an analog-to-digital converter (ADC) or digital-to-analog converter (DAC)), a signal processor (e.g., filter, amplifier, etc.), a recorder, and/or a user interface (e.g., display, touchscreen, keyboard, etc.). Moreover, the device 16 can comprise one or more electrical components for establishing wired or wireless communication with other electronic devices (e.g., a PC or external power supply). For instance, the device 16 can comprise terminals and/or leads for establishing wired connections, or a transponder for establishing wireless communication. Broadly speaking, the device 16 can comprise any configuration of one or more electrical components that can electrically communicate with the nerve 14 via the nerve interface 12.

In some examples, the system 10 can further include a sensor 40 that is configured to detect a neuropathological condition of the subject. For example, the sensor 40 can be an O₂ sensor, a pressure sensor, a motion (e.g., acceleration or displacement) sensor, an EKG, a strain gauge, or some other sensor that is configured to detect a condition of the subject that relates (or potentially relates) to a neuropathology. As another example, the sensor 40 may be another nerve interface that is configured to detect neuroactivity of a different nerve and provide an electrical signal corresponding to the neuroactivity. Broadly speaking, the sensor 40 can be any device configured to sense a condition of the subject that is indicative (or potentially indicative) of the presence or absence of a neuropathology.

As discussed further below, the electronic device 16 can be operated to apply electrical stimulus to the nerve 14 (via the interface 12) based on feedback from the sensor 40. In some examples, the sensor 40 can be operatively coupled with a microprocessor of the electronic device 16, which can automatically apply the electrical stimulus based on the feedback. Alternatively, a user can monitor feedback from the sensor 40 and manually operate the electronic device 16 to apply the electrical stimulus.

The system 10 may also include a neuropathology treatment device 50 that can be operated to treat a neuropathological condition of the subject. The treatment device 50 can be, for example, a CPAP machine, a BiPAP machine, a ventilator, an artificial diaphragm, or an anesthesia system. As discussed further below, the treatment device 50 can be operated based on neuroactivity detected by the interface 12. In some examples, the treatment device 50 can be operatively coupled with a microprocessor of the electronic device 16, which can automatically operate the treatment device 50 based on the detected neuroactivity. Alternatively, a user can monitor the neuroactivity and manually operate the treatment device 50 based on the neuroactivity.

The system 10 as described above can be used to monitor neuroactivity and/or apply electrical stimulus to the nerve 14 for the purposes of treating (e.g., monitoring, diagnosing, preventing, mitigating, etc.) neuropathologies.

For instance, in one example, neuroactivity of the nerve 14 can produce an electrical signal that is transmitted to the electronic device 16 via the nerve interface 12 and corresponds to the neuroactivity. Meanwhile, the sensor 40 can be operated to detect a neuropathological condition of the subject of provide an electrical output indicative of the detected condition. That output can be compared with the electrical signal of the nerve interface 12 to determine if there is any correlation between neuroactivity of the nerve 14 and the neuropathological condition.

In another example, the treatment device 50 can be operated based on the electrical signal transmitted to the electronic device 16 via the nerve interface 12. For instance, a microprocessor of the electronic device 16 can compare the electrical signal with a predetermined threshold to determine if a neuropathological condition is present. If the comparison indicates that a neuropathological condition is present, the microprocessor can operate the treatment device 50. Conversely, if the comparison indicates that a neuropathological condition is not present, the microprocessor can cease or refrain from operating the treatment device 50.

In another example, the microprocessor of the electronic device 16 can apply an electrical stimulus to the nerve 14 via the nerve interface 12. Meanwhile, the sensor 40 can be operated to detect a neuropathological condition of the subject or provide an electrical output indicative of the detected condition. That output can be compared with the electrical stimulus applied to the nerve 14 to determine if there is any correlation between neurostimulation of the nerve 14 and the neuropathological condition.

In another example, the microprocessor of the electronic device 16 can apply an electrical stimulus to the nerve 14 based on a neuropathological condition detected by the sensor 40. More specifically, the microprocessor of the electronic device 16 can compare the electrical output of the sensor 40 with a predetermined threshold to determine if a neuropathological condition is present. If the comparison indicates that a neuropathological condition is present, the microprocessor can apply an electrical stimulus to the nerve 14 via the nerve interface 12. Conversely, if the comparison indicates that a neuropathological condition is not present, the microprocessor can cease or refrain from applying an electrical stimulus to the nerve 14.

The system 10 as described above can be used to monitor neuroactivity and/or apply electrical stimulus to the nerve 14 for the purposes of treating (e.g., monitoring, diagnosing, preventing, mitigating, etc.) neuropathologies. Another benefit of the system 10 is to produce a regenerative peripheral nerve interface (RPNI) within the neck to augment a signal from designated neural activity from the cranial nerves. In particular, the nerve interface 12 comprises an electrode with an electro-conductive polymer that can be used to coat a nerve-muscle pedicle flap or targeted regenerative nerve into a vascularized muscle for monitoring of motor function. The polymer can pick up that signal and convert it to an electronic signal, which would either be connected to a stimulatory or neuromodulation implant to control another target muscle site or prosthesis.

This approach is beneficial because other solutions place electrodes on the surface of the skin or the nerve, suturing small wires into the nerve or penetrating the nerve with needles. These are short-term solutions because the nerves are delicate and build scar tissue over time. The nerve interface 12 of the present disclosure, however, can provide a regenerative peripheral nerve interface (RPNI) to transduce neural signals and provide high-fidelity of signals without penetrating or otherwise mutilating the nerve.

Illustrative embodiments have been described, hereinabove. It will be apparent to those skilled in the art that the above apparatuses and methods may incorporate changes and modifications without departing from the general scope of this disclosure. The disclosure is intended to include all such modifications and alterations disclosed herein or ascertainable herefrom by persons of ordinary skill in the art without undue experimentation.

1. A method of treating an animal subject, the method comprising:
applying a nerve interface to a muscle pedicle of the subject, the muscle pedicle having a peripheral nerve inserted therein, wherein the nerve interface is configured to provide electrical communication between the peripheral nerve and an electronic device.

2. The method of claim 1, further comprising dissecting a portion of a muscle of the subject to yield the muscle pedicle, and inserting the peripheral nerve therein.

3. The method of claim 2, wherein the peripheral nerve comprises a cranial nerve or cervical nerve.

4. The method of claim 2, wherein the peripheral nerve comprises an ansa hypoglossi nerve, and the muscle comprises a stemohyoid muscle.

5. The method of claim 1, further comprising applying an electrical stimulus to the peripheral nerve via the nerve interface.

6. The method of claim 5, further comprising detecting a neuropathological condition of the subject, wherein the electrical stimulus is applied to the peripheral nerve based on the neuropathological condition.

7. The method of claim 1, wherein the electronic device receives an electrical signal from the nerve interface, the electrical signal corresponding to neuroactivity of the peripheral nerve.

8. The method of claim 7, further comprising operating a neuropathology treatment device based on the electrical signal.

9. The method of claim 1, wherein the nerve interface comprises:
a main body,
an electrode affixed to the main body, and
one or more conductive polymers that establish electrical communication between the peripheral nerve and the electrode.

10. A method of treating an animal subject, the method comprising:
applying a nerve interface to a cranial or cervical nerve, wherein the nerve interface comprises:
a main body,
an electrode affixed to the main body, and
one or more conductive polymers that establish electrical communication between the peripheral nerve and the electrode, and
monitoring neuroactivity of the cranial or cervical nerve, or applying electrical stimulus to the cranial or cervical nerve, via the nerve interface.

11. The method of claim 10, wherein the nerve interface is applied to a muscle pedicle of the subject, the muscle pedicle having the cranial or cervical nerve inserted therein.

12. A nerve interface device for treating a subject comprising an electrical interface configured to be applied to a muscle pedicle of an animal subject, the muscle pedicle having a peripheral nerve inserted therein, wherein the nerve interface is configured to provide electrical communication between the peripheral nerve and an electronic device.

13. The nerve interface device according to claim 12, wherein the peripheral nerve is a cranial or cervical nerve and wherein is the electrical interface is configured to provide electrical communication between the cranial or the cervical nerve and the electronic device.

14. The nerve interface device according to claim 13, for use in monitoring of neuroactivity and/or stimulation of the peripheral nerve.

15. The nerve interface device according to claim 14, for use in the treatment of sleep apnea.

16. The nerve interface device according to claim 15, wherein the peripheral nerve comprises an ansa hypoglossi nerve, and the muscle comprises a sternohyoid muscle.

17. The nerve interface device according to claim 16, wherein it is further configured to apply an electrical stimulus to the peripheral nerve via the electrical interface.

18. A system comprising the nerve interface device according to claim 17, and an electronic device configured to receive an electrical signal from the nerve interface, the electrical signal corresponding to a neuro signal of the peripheral nerve.

19. The nerve interface device according to claim 17, wherein the nerve interface comprises:
a main body,
an electrode affixed to the main body, and
one or more conductive polymers configured to establish electrical communication between the peripheral nerve and the electrode.

20. A system for treating sleep apnea of an animal subject comprising:
the nerve interface device according to claim 17, and
an electronic device;
wherein the system is configured to monitor neuroactivity of a peripheral nerve, preferably the peripheral nerve being a cranial or cervical nerve, of the subject and/or to apply electrical stimulus to a cranial or cervical nerve of the subject.
